(19) United States
(12) Patent Application Publication
(10) Pub. No.: US 2021/0161461 A1
(43) Pub. Date: Jun. 3, 2021

(54) SYSTEM AND METHOD FOR CERVICAL CANCER SCREENING

(71) Applicant: DALRADA HEALTH PRODUCTS INC., Escondido, CA (US)

(72) Inventors: Payal KESWARPU, Bengaluru (IN); David PICKET, Escondido, CA (US)

(73) Assignee: DALRADA HEALTH PRODUCTS INC., Escondido, CA (US)

(21) Appl. No.: 16/780,728

(22) Filed: Feb. 3, 2020

(30) Foreign Application Priority Data
Nov. 28, 2019 (IN) 201941048898

(51) Int. Cl. A61B 5/00; A61B 5/145; A61B 1/303; A61B 1/32; A61B 42/10; A61B 50/30; A61B 46/20; A61F 13/38; A61B 90/94; A61B 90/96; A61B 1/00; A61K 49/00

(52) U.S. Cl. CPC A61B 5/4331; A61B 2562/0295; A61B 1/303; A61B 1/32; A61B 42/10; A61B 50/30; A61B 46/20; A61F 13/38; A61B 90/94; A61B 90/96; A61B 5/7264; A61B 1/00011; A61B 1/00009; A61K 49/0004; A61B 5/14539

(57) ABSTRACT

A system and method for screening cervical cancer is disclosed. The system and method enable screening of cervical cancer locally at geographical location of subjects using minimum infrastructure. The system includes a visual inspection kit, a device, and a subject connection module. The visual inspection kit may include a vital stain having a predefined concentration, a sterile accessory, and an applicator accessory. The device may include an application for data collection, storage, transmission, retrieval and analytics and the subject connection module is configured to manage interaction with subjects. The method includes screening a subject using the visual inspection kit, storing and analyzing the images using the device, and connecting with subject using the subject connection module.

This patent application claims priority from IN Provisional Application number 201941048898 filed on Nov. 28, 2019, which is incorporated by reference herein.

### FIELD OF THE INVENTION

The present disclosure relates to a system and a method for screening for cervical cancer. More particularly, the present disclosure relates to a comprehensive solution for screening a subject for cervical cancer.

### BACKGROUND OF THE INVENTION

Cervical cancer is the second most common cancer in women worldwide. More than three-fourths of the cervical cancer subjects are diagnosed, for the first time, at advanced stages of the cancer leading to poor prognosis of long-term survival and cure. Screening for cervical cancer aims to detect the disease at a precancerous stage when it is amenable to treatment and cure. Generally used methods for cervical cancer screening are Pap smear (also known as cytology based) test, Human Papilloma Virus (HPV) testing, and visual inspection methods. Though cytology based or virus detection based screening provide effective information in the precancer stages, limited local infrastructure and resources may hinder wide use of these screening methods.

Visual inspection-based approaches to cervical cancer screening demands minimum infrastructure. Use of unaided visual inspection (that is, without the use of vital stains or dyes) may suffer from low sensitivity and low specificity to detect cervical neoplasia, particularly the precursor lesions. Aided visual inspection using vital stains may provide valuable screening information about the cervical cancer.

Aided visual inspection procedure needs rigorous quality control in terms of preventing cross infection and undesired chemical interaction. For example, reuse of unsterilized equipment can spread the infection. Further, variations in the concentration of the vital stain used in the aided visual inspection procedure may be harmful to the subject or result in variation in the results of the screening. For example, if the vital stain is too concentrated, it may cause chemical burn and if the stain is too dilute, the reaction on tissue would not be evident and may lead to false negative results. Besides the procedure related issues, other challenges prevail around delivering results and following up cases after the test is carried out. In some cases, an expert may be needed to review and interpret the results. Further, currently the subject needs to travel to a nearby hospital for the screening and for receiving the interpreted results after a few days. Therefore, there is a need for a comprehensive, easy, and robust screening of cervical cancer demanding the least possible infrastructure and simplified methods of screening, interpretation of results, and communicating the results to the subject.

### BRIEF SUMMARY OF THE INVENTION

This summary is provided to introduce a selection of concepts in a simple manner that is further described in the detailed description of the disclosure. This summary is not intended to identify key or essential inventive concepts of the subject matter nor is it intended for determining the scope of the disclosure.

In one aspect of the present disclosure, a system for screening a subject for cervical cancer is disclosed. The system includes a visual inspection kit and a device. The visual inspection kit includes a vital stain having a predefined concentration, a sterile accessary, and an applicator accessory. The device is configured for at least one operation selected from the group of collecting, storing, retrieving, transmitting, and analyzing of data. The data comprises at least one image of a cervical epithelium of the subject.

In another aspect of the present disclosure, a method for screening a subject for cervical cancer is disclosed. The cervical epithelium of the subject has been treated with a vital stain prior to using the method. The method includes capturing one or more images of the cervical epithelium of the subject and analyzing the captured images using a device. The device classifies each of the one or more images as that of indeterminate, non-cancerous, pre-cancerous, or cancerous cervical epithelium.

### BRIEF DESCRIPTION OF THE FIGURES

The disclosure will be described and explained with additional specificity and detail with the accompanying figures in which:

FIG. 1 illustrates components of a cervical screening system communicating with a subject and an expert, in accordance with one embodiment of the present disclosure; and

FIG. 2 shows contents of an exemplary visual inspection kit, in accordance with one embodiment of the present disclosure.

Further, skilled artisans will appreciate that elements in the figures are illustrated for simplicity and may not have necessarily been drawn to scale. Furthermore, in terms of the construction of the device, one or more components of the device may have been represented in the figures by conventional symbols, and the figures may show only those specific details that are pertinent to understanding the embodiments of the present invention so as not to obscure the figures with details that will be readily apparent to those of ordinary skill in the art having the benefit of the description herein.

### DETAILED DESCRIPTION

It will be understood by those skilled in the art that the foregoing general description and the following detailed description are exemplary and explanatory of the disclosure and are not intended to be restrictive thereof.

The terms “comprises”, “comprising”, or any other variations thereof, are intended to cover a non-exclusive inclusion, such that a process or method that comprises a list of steps does not include only those steps but may include other steps not expressly listed or inherent to such a process or a method. Similarly, one or more elements or structures or components preceded by “comprises . . . a” does not, without more constraints, preclude the existence of other elements, other structures, other components, additional devices, additional elements, additional structures, or additional components. Appearances of the phrase “in an embodiment”, “in another embodiment” and similar language throughout this specification may, but do not necessarily, all refer to the same embodiment.

Unless otherwise defined, all technical and scientific terms used herein have the same meaning as commonly understood by one of ordinary skill in the art to which this disclosure belongs. The components, methods, and examples provided herein are illustrative only and not intended to be limiting. As used herein, a “subject” is an individual undergoing the screening for cervical cancer, hence does not necessarily mean a person already suffering from cancer or other diseases.

The present disclosure relates to a system and a method for providing a preliminary diagnosis of cervical cancer or an assessment of cervical cancer risk. The method can be used for screening a subject for cervical cancer. The screening of the subject for cervical cancer may be conducted for (i) identifying whether cancerous cells are present in the cervical epithelium, (ii) if the cancerous cells are not present, whether there is any symptoms to indicate possible risk of developing cervical cancer in the near future, (iii) if the cancerous cells are present, identifying a stage of cancer. This kind of screening, if carried out on larger population, may be as a part of public drive or as a part of primary health diagnosis, can give valuable information regarding the care that need to be taken by the subject to control and/or cure the cancerous growth or to avoid developing cancer. Simplification in the screening methods would encourage accessibility of the subject for the screening, thus enhancing an early detection and awareness.

The system disclosed herein includes a visual inspection kit that contains all the all the necessary materials and accessories needed by a primary healthcare worker for screening a subject for the cervical cancer. The visual inspection kit comprises a vital stain having a predefined concentration, a sterile accessary, and an applicator accessory. The vital stain is applied, by a primary healthcare worker, to the epithelium of the subject using the sterile accessory and the applicator accessory. The vital stain may be applied by topically smearing the cervical tissue uniformly with the vital stain or by spraying to the cervical area. At least one image of the cervical epithelium of the subject is captured. The method functions by verifying an absence of cancer risk or identifying pre-cancerous or cancerous cells by analyzing the at least one captured image. The results may be validated by an expert.

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications to the disclosure, and such further applications of the principles of the disclosure as described herein being contemplated as would normally occur to one skilled in the art to which the disclosure relates are deemed to be a part of this disclosure.

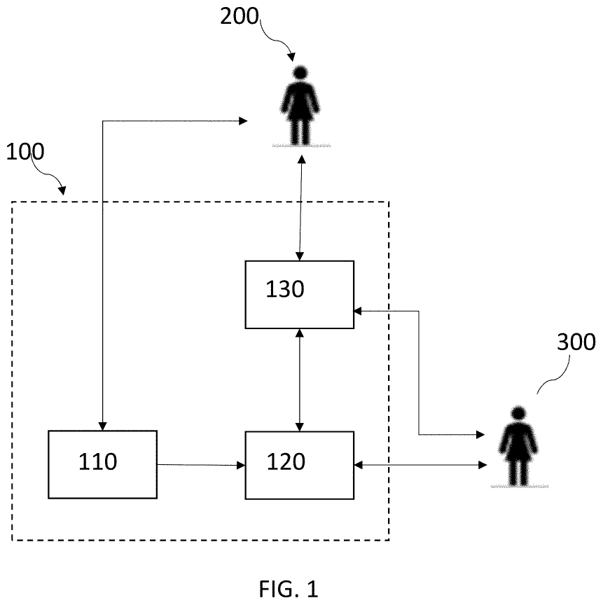

FIG. 1 illustrates a schematic diagram of an exemplary system 100 used for cervical cancer screening of a subject 200. The system 100 includes a visual inspection kit 110 that can be used locally at or near to the geographical location of the subject 200, without a need for the subject 200 to visit an established laboratory for the screening. The visual inspection kit 110 includes accessories necessary for a safe and systematic collection of lesion details of the epithelium of the subject 200 for inspection using an aided visual inspection method. An expert 300 may be consulted for further analyzing the results of the visual inspection method.

The visual inspection kit 110 includes a vital stain, at least one sterile accessory, and at least one applicator accessory. The vital stain is an active element of the visual inspection kit 110. A vital stain is a dye used to stain living tissue for visual or microscopical observation. Vital stains, at appropriate dilution, are harmless to living tissue. Various kinds of vital stains may be used alone, or in combination with one or more vital stains or any other augmenting chemicals as part of the visual inspection kit 110. The vital stain in the visual inspection kit 110 may include one or more vital stains from the list of vital stains including acetic acid, Lugol's iodine, fluorescein sodium, methylene blue, toluidine blue, and Rose Bengal. In some embodiments, the vital stain is in the form of an aqueous solution. The effective and adequate concentration ranges of different vital stains may vary from one another. In some embodiments, acetic acid is used as vital stain in the visual inspection kit 110 for cervical cancer screening.

Acetic acid reacts with cells of the epithelium and causes a reversible coagulation or precipitation of the nuclear proteins and cytokeratins. Thus, the effect of acetic acid depends upon the amount of nuclear proteins and cytokeratins present in the epithelium. When acetic acid is topically applied to normal squamous epithelium, little coagulation occurs in the superficial cell layer, as this is sparsely nucleated. Pre-cancerous and cancerous areas, areas healing from ulcers, etc., undergo maximal coagulation due to their higher content of nuclear protein and prevent light from passing through the epithelium. As a result, the subepithelial vessel pattern is obliterated and becomes less easy to see and the epithelium appears white in the case of using acetic acid as the vital stain. This reaction is termed aceto-whitening and produces a noticeable effect when compared to the normal pink color of the surrounding normal squamous epithelium of the cervix, an effect that is commonly visible to the naked eye. Other vital dyes similarly interact with the cells and indicate, primarily by change in color or intensity of the color or both, in areas where abnormality exists.

The vital stain included in the visual inspection kit 110 has a predefined concentration. As used herein, a “predefined concentration” is a selected concentration that is decided to be included in the visual inspection kit 110. The predefined concentration may be selected based on one or more factors associated with the vital stain, the diluent used for diluting, the ease and effectiveness of diluting the vital stain using the diluent, and so on.

In some embodiments, the predefined concentration of the vital stain may be pre-adjusted for use without the need for any operations on it and without any further need to change the concentration. In these embodiments, the predefined concentration is the concentration that is suitable for applying to the epithelium directly, without undergoing any further processing such as diluting before application. The suitable concentration may be determined based on the safety of the subject and effective concentration range of the vital stain. The effective concentration range of the vital stain may be a range of concentration where the results obtained by the use of that vital stain are reliable, reproducible, accurate, and can be effectively converted into identifiable symptoms or conditions. For example, the vital stain may have to be of sufficient concentration to promote adequate reaction with the epithelium cells and produce noticeable change in color or intensity or both to obtain reliable analysis of the color change. Further, the concentration may need to be limited to reduce or avoid chemical burns in the subject. Therefore, a specific and advantageous concentration may be identified as the predefined concentration for the vital stain that is to be included in the visual inspection kit 110 for use without further processing. In an embodiment using acetic acid as the vital stain, an aqueous solution of acetic acid is used with a directly usable predefined concentration that is in a range from 2% volume/volume (referred hereinafter as v/v) to 10% v/v. In an exemplary embodiment, 5% v/v aqueous acetic acid is included as the vital stain in the visual inspection kit 110 and is used for the screening directly.

In some embodiments, the predefined concentration is any concentration that may be modified to the required concentration for applying, immediately prior to applying it to the epithelium. For example, the predefined concentration may be higher than the concentration of the vital stain required for the use on the epithelium cells at the time of screening. In these conditions, the required concentration for applying may be achieved by diluting the vital stain, prior to applying. One or more diluents may be provided in the visual inspection kit 110 for carrying out this step. For example, the visual inspection kit 110 may include, a diluent, such as distilled water, to be added to the vital stain to establish the concentration to the value required for applying. Depending on the predefined concentration of the vital stain included in the visual inspection kit 110 and the required concentration for applying, a precise quantity of diluent may be provided as a part of the visual inspection kit 110. The presence of precise quantity of diluent ensures accurate dilution from the predefined concentration to the required concentration, by avoiding any errors in diluting, data collection and analyzing. In some other embodiments, the predefined concentration may be lower than the required concentration for applying to the epithelium. (and a precise quantity of a concentrated vital stain is provided separately?)

Prior experiments may be carried out using one or more individual concentrations of the vital stain in the advantageous concentration range on selected multiple non-cancerous, suspicious, precancerous, and cancerous cell samples and a library of data may be created for each standardized individual concentration. The library of data may include associating the individual concentration with the expected data for the cancerous, suspicious, precancerous, and non-cancerous cells treated with the individual concentration. This library may be used to identify and analyze the data collected during screening the subject.

During screening, the subject undergoes a procedure in which the epithelium cells of the subject are topically applied with the vital stain having a suitable concentration. Images of the epithelium cells after applying the vital stain are captured for identifying the state of the cells. It may be necessary to wait a predefined time after the topical application of the vital stain before the images are captured. At least one sterile accessory and at least one applicator accessory are included in the visual inspection kit 110 to enable applying the vital stain to the epithelium of the subject. A sterile accessory may be an accessory that is previously sterilized and directly usable for a process during visual inspection. Any process that requires a direct contact with the epithelium may include using a sterile accessory. For example, in one embodiment, a set of sterilized nitrile impermeable gloves, a disposable sterile speculum, or a combination of the two are included in the visual inspection kit 110 as sterile accessory and used while applying the vital stain to the epithelium of the subject 200. In some other embodiments, the sterile accessory may be an accessory to sterilize one or more tools used in the inspection process. For example, a sterilizer kit may be included in the visual inspection kit 110 and used for sterilizing the required accessories immediately prior to using for screening. Non-limiting examples of a sterile accessory that may be used for sterilizing a tool to be used in the visual inspection may include a sanitizer, an ultraviolet source, an infrared source, denatured spirit, etc.

The applicator accessory may include any accessory that is used for or aiding a substantially uniform application of the vital stain to the epithelium or for cleaning the epithelium tissue cells before or after applying the vital stain. In some embodiments, the applicator accessory includes at least one accessory selected from a group of accessories comprising a cotton swab, a clean drape, and a saline solution. In the above embodiments, the applicator accessory may include any one of or a combination of the sterile cotton swab, drape, and saline solution.

The “data” as used herein may be any information that is associated with the screening of the subject. For example, the data may include any information regarding the subject, geographical location of the screening, the unique identifier of the visual inspection kit, procedure conducted on the subject, results obtained from the screening, etc. The data includes at least one image obtained from the visual inspection of the epithelium of the subject after applying the vital stain. The image of the epithelium cells may be captured using any analog or digital imaging device including, but not limited to, a camera, a mobile phone, or a scanner. In one embodiment, the image is captured using a radiation in the visible wavelength range. In some embodiments, an image may be captured using one or more of ultraviolet (UV) imaging, UV fluorescence imaging, or infrared (IR) imaging in addition to, or instead of, visual wavelength range imaging.

The dilution of the vital stain is desirable to be substantially accurate to obtain consistent results for image analysis. Considering the significance of having the right concentration of the vital stain for the successful examination process, the visual inspection kit 110 may contain one or more accessories for measuring the concentration of the vital stain. One accessory for measuring the concentration may be a concentration indicator. The concentration measurement of the vital stain may be used as a verification process to ensure precise concentration that is targeted to be used in the screening. For example, the predefined concentration of the vital stain in the visual inspection kit 110 may be verified at the time of screening and any difference in concentration may be corrected prior to the application of the vital stain. In the embodiments having instant dilution step, the predefined concentration, final concentration, or both may be verified using the concentration indicator.

In some embodiments, a pH indicator may be used as a concentration indicator and included in the kit to ascertain the right concentration of the vital stain. A predefined pH chart of various concentrations of the vital stain may be maintained and used as a measure to verify the desired concentration of solution. The formula

pH=−log(\sqrt{c·Ksp})

may be used to determine concentration (c) of the solution, where Ksp indicates the solubility product constant of the solution. The pH indicator may prevent false negative results due to additional dilution and undesired side effects to subject 200 due to higher concentrations. The pH indicator may be a pH strip, a bar meter or any other device or accessory that can be used for simple and a sufficiently accurate measurement of pH of the vital stain before dilution, after dilution, or at both the instances. Smart phone-based pH meters may also be used for the concentration and/or pH measurement optionally using any of the support accessories that may be present as part of the visual inspection kit 110.

The system 100 also includes a device 120 configured for at least one operation selected from a group of operations of collecting, storing, retrieving, transmitting, and analyzing. The device 120 may be a portable device or a centrally located, non-portable device. A portable device 120 may be a hand held device such as a mobile phone or a PDA, or a smartphone, or a tablet and so on that may be carried along by a primary healthcare worker, for instance, from place to place without undue effort or strain or inconvenience. A non-portable device 120 may be used from a centralized location also, without being carried to the geographical location of screening, yet used for receiving the data of visual inspection. In an example embodiment, the device 120 may digitally receive the one or more images collected by the healthcare worker using the visual inspection method. In some embodiments, the device 120 is configured for storing the collected data in a retrievable way, analyzing the data, and transmitting the collected or analyzed data or both to the expert 300 or to another system 100 as per the requirement. The device 120 may be a computer, a tablet device, mobile phone, or any other digital resource. The device 120 need not be a dedicated device for this application. The device 120 may also be used to enable linking brief clinical history, examination findings, and other patient records. The device 120 may have an application feature to select specific images from a bunch of images stored in the device to enable analyzing a wide variety of images.

The data collected may be reviewed by the expert 300 for any abnormality. The expert 300 may review the data locally or through remote consultation. The data may be electronically transferred to the expert 300 for the consultation. In some embodiments, the electronic transfer of data is carried out through the device 120. For example, if the device 120 is a portable device that is used by the healthcare worker at the geographical location of the screening, the data may be transferred from the device 120 to another device (not shown in FIG. 1) that is accessible by the expert 300. In some embodiments, the device 120 is directly accessible by the expert 300 and the data from the screening is electronically transferred to the device 120 by the healthcare worker. In yet another embodiment, functions such as data capture, analyzing, and accessing by the expert are carried out using different devices. Further, intelligent image processing-based algorithms may be utilized to triage the case based on factors such as acetowhite kinetics, Lugol's iodine reaction etc.

The visual inspection kit 110 may further have a means for uniquely identifying the kit. The means for unique identification may be a human readable alphanumeric identifier, a machine-readable alphanumeric identifier, or a combination of the two. For example, the kit may have a serial number printed on it facilitating its unique identification. In another example embodiment, the kit 110 may include a barcode or a QR code. The barcode may further include other information of the kit such as date of manufacturing, date of expiry, production or manufacture lot information etc. The unique identification may be linked with the subject 200 to ensure error free labelling of each subject's findings in connection with the individual visual inspection kit 110 used for that subject 200. In some embodiments, the visual inspection kit 110 is a single use, disposable kit, thus maintaining hygiene and specificity of the kit to each subject 200. The findings may be stored in the device 120 using this identification number along with identification details of the subject 200. The captured one or more images may be linked to the unique identification number of the visual inspection kit 110 and may be analyzed, stored, or transferred as required. The images may be retrievably stored in the device 120 to refer to or recall in later stages, as and when required.

The visual inspection method may be carried out by an individual trained to carry out the method, without the need for presence of an expert in the medical profession at the site of carrying out the visual inspection. For example, a trained nurse or primary healthcare provider may be able to conduct the screening without the presence of a gynecologist or a pathologist at the site of the inspection. The images obtained may be analyzed by the trained professional and may be shared with an expert 300 in the field for further analysis or for obtaining a diagnosis. In an example embodiment, using the images, the lesions, if any, may be differentiated into normal, cancerous, or suspicious lesions. The images may also be digitally shared with the expert 300 in a geographical location other than where the screening or visual inspection is carried out and the analysis or the opinion may be obtained instantly without any inordinate delay in obtaining the screening results.

In another embodiment, the individual or the subject 200 may be able to follow the procedure, collect images and send them along with the unique identification of the visual inspection kit 110 and the subject 200 for analysis, without even a need of a trained professional for the visual inspection. The individual may receive the required instructions through mass or individual education—direct or online—or through an instruction note that may be included in the visual inspection kit 110.

The device 120 may further store important demographic information including unique country specific ID to enable linking other subject records, brief clinical history and examination findings. Further, while registering a subject 200, the unique identification of the kit, such as a bar code may be scanned and the data pertaining to the kit 110 and subject identifier are matched. This would aid in traceability of the subject 200 and provides option to maintain a record of the visual inspection kit 110 that is used in the procedure. The application may have features to capture images and store in the device. The data collected may be retrieved and reviewed for any abnormality and/or transmitted for remote consultation. Retrieval may be in the form of an electronic output or a printout. The data may be stored and handled securely with decentralized control and maintained in a form that is tamper-resistant and complies with information auditing guidelines based on the principle of separation of duties and consequent selective access. Intelligent image processing-based algorithms may also be utilized to categorize the images based on reaction factors of the vital stain such as acetowhite kinetics, Lugol's iodine reaction, etc.

The system 100 may further include a subject connection module 130 that can facilitate and manage interactions with the subject 200. The interactions with the subject 200 may be made before or after collection of images of the epithelium cells. Subject report sharing, suggestions, and follow up may be carried out by interacting with the subject connection module 130. The subject connection module 130 may be used by the system 100 to telephonically or electronically connect with the subject's device, or a device the subject 200 has access to, and digitally send and receive messages and reports. The subject connection module 130 may be used to educate the subject 200 before or after screening, communicate the results of screening to the subject 200, advice the subject 200 on the action to be taken by the subject 200, remind about the action or actions due, schedule a follow up, facilitate subject's interaction with a health expert 300 etc. For example, report for each visit of the subject 200 to the health provider, procedures carried out in the visit, and the results of the procedures, recommendations of the doctor during the visit etc., can be sent to the subject through subject connection module 130 as messages, email attachment, or in the form of a printout. The subject connection module 130 may also be used to collate tests and treatment particulars conducted at different centers or geographical locations in a Health Insurance Portability and Accountability Act (HIPAA) compliant manner. In some embodiments, the subject connection module 130 is in communication with the device 120 and with the expert 300. In some embodiments, the subject connection module 130 is in communication with the expert 300 through the device 120. In some embodiments, the subject 200 may also directly communicate with the expert 300 (not shown in FIG. 1).

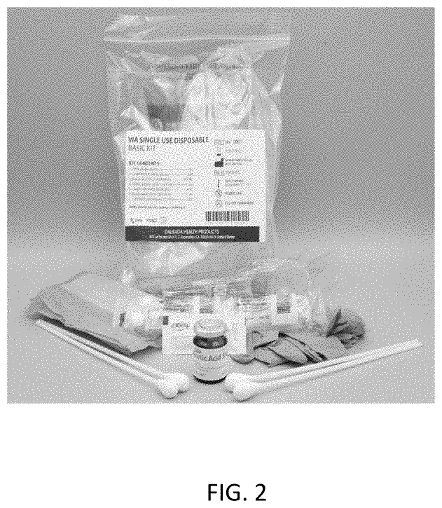

FIG. 2 shows components of a non-limiting, example visual inspection kit 110. The kit 110 includes 5% (v/v) aqueous solution of acetic acid, a pH meter, distilled water, 0.9% saline solution, a clean drape, sterile nitrile gloves, a cotton tip applicator or swab and disposable sterile plastic speculum. The pH meter has a resolution of 0.01 and can be used to verify the concentration of the solution. The example visual inspection kit 110 has a unique barcode specific to the kit.

An example method of screening a subject for cervical cancer using the visual inspection kit 110 of FIG. 2 is described below. The kit may be opened in the geographical location of screening. The kit may be opened, and the pH meter may be used to confirm the concentration of the acetic acid solution. The sterile gloves may be used by the healthcare provider during the procedure. The sterile speculum may be used to enable visibility of the cervix area that is to be examined. The speculum may be lubricated with water or the saline solution and pressed against the cervix to expose the cervix area. The area of inspection may be wiped with a dry cotton swab to remove any discharge, blood or mucus. A cotton swab soaked in the 5% v/v aqueous acetic acid should liberally be applied to the cervix. After a waiting period of about 1-2 minutes, the epithelium may be observed for any color change such as acetowhitening. Multiple digital images can be taken using a mobile camera capturing the details such as the location of the acetowhitened areas, extension, intensity of whiteness, borders and demarcations, as well as size. The images may be analyzed using the mobile phone or any other device and the subject may be apprised of the results and requirement of further follow up or procedure, if required.

The present disclosure provides a comprehensive solution for screening cervical cancer in a large population without the need for large trained workforce or the necessity of long travel of the subjects. Sterilized and single use units are designed for maintaining quality, accuracy and easy accessibility.

While specific language has been used to describe the disclosure, any limitations arising on account of the same are not intended. As would be apparent to a person skilled in the art, various working modifications may be made to the method in order to implement the inventive concept as taught herein. For example, the visual inspection kit 110, device 120, or subject connection module 130 may be individually used without linking with one another, but the synergistic effect of using them in combination provides for the simplicity and reach of the cervical cancer screening system 100.

The figures and the foregoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, orders of processes described herein may be changed and are not limited to the manner described herein. Moreover, the actions of any flow diagram need not be implemented in the order shown; nor do all of the acts necessarily need to be performed. Also, those acts that are not dependent on other acts may be performed in parallel with the other acts. The scope of embodiments is by no means limited by these specific examples. Numerous variations, whether explicitly given in the specification or not, such as differences in structure, dimension, and use of material, are possible. The scope of embodiments is at least as broad as given by the following claims.

1. A system for use in screening a subject for cervical cancer, the system comprising:
a visual inspection kit comprising a vital stain having a predefined concentration, a sterile accessory, and an applicator accessory; and
a device configured for at least one operation selected from a group of operations of collecting, storing, retrieving, transmitting, and analyzing of data, wherein the data comprises at least one image of a cervical epithelium of the subject.

2. The system as claimed in claim 1, comprising a subject connection module configured for facilitating interactions with the subject.

3. The system as claimed in claim 1, wherein the predefined concentration is a concentration that is suitable for applying to the cervical epithelium.

4. The system as claimed in claim 1, wherein the predefined concentration is a concentration that is modifiable to the suitable concentration by the addition of a diluent.

5. The system as claimed in claim 1, wherein the visual inspection kit comprises a concentration indicator for indicating the concentration of the vital stain.

6. The system as claimed in claim 5, wherein the concentration indicator is a pH indicator comprising at least one pH indicator selected from a group of pH indicators comprising a pH strip, a pH meter, and an accessory for measuring a pH value of the vital stain.

7. The system as claimed in claim 1, wherein the vital stain comprises at least one vital stain selected from a group of vital stains comprising acetic acid, Lugol's iodine, fluorescein sodium, toluidine blue, methylene blue, and Rose Bengal.

8. The system as claimed in claim 1, wherein the sterile accessory comprises at least one sterile accessory selected from a group of sterile accessories comprising a sterile glove, a sterile speculum, and a sterilizer kit.

9. The system as claimed in claim 1, wherein the applicator accessory comprises at least one applicator accessory selected from a group of applicator accessories comprising sterile cotton swab, a drape, and a saline solution.

10. The system as claimed in claim 1, wherein the visual inspection kit comprises at least one means for uniquely identifying the kit, selected from a group of means comprising a human readable alphanumeric identifier and a machine readable alphanumeric identifier.

11. A system for screening a subject for cervical cancer, the system comprising:
a visual inspection kit comprising an aqueous solution of acetic acid of 5% volume/volume concentration, a sterile nitrile glove, and a cotton swab; and
a device configured for collecting, storing, retrieving, transmitting, and analyzing of data, wherein the data comprises at least one image of a cervical epithelium of the subject, treated with the aqueous solution of acetic acid of 5% volume/volume concentration.

12. A method for screening a subject for cervical cancer, wherein the cervical epithelium of the subject has been treated with a vital stain, the method comprising:
capturing one or more images of the cervical epithelium of the subject; and
analyzing each of the one or more images using a device configured for classifying each of the one or more images as one of indeterminate, non-cancerous, pre-cancerous, and cancerous cervical epithelium.

13. The method as claimed in claim 12, further comprising interacting with the subject using a subject connection module.
